(19) United States
(12) Patent Application Publication
(10) Pub. No.: US 2003/0148017 A1
(43) Pub. Date: Aug. 7, 2003

(54) Copolymer coating for a hydrophobic membrane

(76) Inventors: Olli Tuominen, Marlboro, MA (US); Eric W. Stroup, North Ogden, UT (US); Chih-Hu Ho, Sandy, UT (US); Justin W. Cook, Houston, TX (US)

Correspondence Address: GIBSON, DUNN & CRUTCHER LLP, Suite 4100, 1801 California Street, Denver, CO, 80202-2641, US

(21) Appl. No.: 10/013,323

(22) Filed: Dec. 7, 2001

(51) Int. Cl. B05D 3/00; C02F 1/44; A61M 37/00

(52) U.S. Cl. 427/2.1; 604/5.01; 210/645

(57) ABSTRACT

A coating material and a method for coating a hydrophobic membrane with a copolymer that contains at least one hydrophilic segment and at least one hydrophilic segment, such as a PEO—PPO—PEO triblock copolymer, so that the surface of the membrane becomes hydrophilic. The hydrophilic coating helps repel biological molecules, thereby reducing the risk that these molecules will adsorb or deposit on the membrane surface. When the copolymers are modified with an active group, ligands can be immobilized on the copolymer through the active group so that specific molecules that will bind with the ligands can be targeted for immobilization on the copolymer coating, thereby improving the efficiency of the removal of specific targets.

### BACKGROUND OF THE INVENTION

Hydrophobic membranes have many practical uses in a variety of areas, including the medical field. For example, hydrophobic membranes can be used in medical procedures to extract or remove wastes from a patient's body fluids. During hemodialysis, a patient's blood comes in contact with many parts of the dialysis system, including the semipermeable membrane inside the dialyzer, and these parts of the system need to be biocompatible. As the blood contacts the membrane, problems can occur if the membrane surfaces are not biocompatible.

Most membrane surfaces have a propensity to adsorb substances or molecules, such as proteins, which can create problems for the patient during dialysis treatment. When blood contacts these membrane surfaces, plasma proteins can be adsorbed or deposited on the surface of the membrane, initiating the coagulation of blood along the membrane surface. This is dangerous for the patient because if coagulated blood is returned to the patient, it can block an artery or vein in the patient and prevent blood from flowing throughout the patient's body.

The deposition of substances or molecules, such as proteins, in the blood along the surface of the membrane varies depending upon the surface properties of the membrane and the composition of the blood. Currently, polysulfone or polyethersulfone membranes are often used in dialyzers because these materials are fairly biocompatible as they respond better than other types of membrane materials to the patient's blood during dialysis. When blood comes in contact with the membrane, it can activate several enzymatic pathways, such as the complement system and the coagulation cascade. In addition, leukocytes and platelets can be activated as well. Unlike cuprophan membranes which have been found to activate the complement system, the polysulfone membrane was developed to help minimize the activation of the complement system.

Other steps can be taken to reduce the risk of blood clotting problems during dialysis. The composition of the patient's blood is usually altered and monitored to reduce the chance of blood clotting during treatment. By thinning the patient's blood, the chance of protein deposition on the membrane surface and other potential complications is reduced. For example, a patient's fluid and electrolyte inlet may be regulated to help reduce the risk of blood coagulation during treatment. In addition, medication, such as the sulfated polysulfone anticoagulant heparin, can be administered to dialysis patients to thin the blood and reduce the chance that blood coagulation will occur during treatment. The inhibitory effect of heparin on blood coagulation is due to the chains of heparin being able to bind with the plasma protein antithrombin III and thrombin to form the thrombin-antithrombin m complex. Some patients with diabetes or angiodysplasia require hemodialysis, but undergo treatment without heparin, using an alternative method of “saline flushing” during treatment. There are several disadvantages to this method such as the inability to maintain a patent circuit in a significant proportion of patients, an increased volume load that would need to be removed with dialysis, and added logistical burdens on dialysis nurses and technicians. While both the administration of heparin and “saline flushing” can help reduce protein deposition on the dialyzer's polysulfone membrane surface, additional safeguards that help reduce the amount of heparin that needs to be administered to patients and further reduce the chance of protein adsorption along the membrane surface would be useful.

The adsorption of substances or molecules, such as proteins, on the surface of the dialyzer membrane has been shown to be a significant cause contributing to these problems. Polysulfone or polyethersulfone membranes have reduced many of these problems because these materials have a low complement activation property. However, the problems have not been eliminated and some adsorption or deposition of substances on the membrane surface can still occur during treatment.

The use of surfactants and other coatings to reduce protein absorption on certain surfaces is known in the art. For example, the following patents have been granted in this field: U.S. Pat. No. 5,728,588 to Caldwell, et al.; U.S. Pat. No. 5,516,703 to Caldwell, et al.; U.S. Pat. No. 5,075,400 to Andrade, et al.; U.S. Pat. No. 5,955,588 to Tsang, et al.; and U.S. Pat. No. 6,087,452 to Stewart, et al. However, none of these patents disclose the use of a copolymer coating on the surface of a hydrophobic membrane.

Therefore, there is still a need for the further prevention of the adsorption and deposition of substances on membrane surfaces. In order to minimize the problems associated with the deposition of substances on the surface of the membrane, there is a need to develop a non-fouling membrane coating that will further reduce adsorption on membrane surfaces and further minimize the problems associated with deposition on membrane surfaces.

### SUMMARY OF THE INVENTION

During end stage renal disease treatment (which includes dialysis, hemodialysis, hemoperfusion and hemodiafiltration), a patient's blood is pumped through a dialyzer wherein substances can be removed from or added to the blood as substances flow to and from the blood across a semipermeable membrane in the dialyzer. Substances in the patient's blood that are too large to pass through the pores of the membrane, such as plasma proteins, can adsorb or deposit on the surface of the membrane creating a surface-induced thrombosis reaction. Membrane materials that help reduce and minimize the adsorption or deposition of substances, such as plasma protein, on the surface of the membrane during dialysis treatment are beneficial to patients because they help reduce the risk of problems occurring to the patient.

The properties of the membrane surface can be altered with surface modification techniques to help reduce the chance of adsorption on the membrane surface, thereby reducing the risk of a dangerous reaction. For example, a coating on the membrane surface can be used to help reduce the adsorption or deposition of substances on the membrane surface during dialysis treatment. Copolymers with at least one hydrophobic segment and at least one hydrophilic segment have been found to attach to the surface of hydrophobic membranes, coating the membrane surface and reducing the adsorption or deposition of substances on the membrane surface. For example, polyethylene oxide (PEO) and polypropylene oxide (PPO) copolymers can be immobilized on the surface of a polysulfone membrane, coating the membrane surface. The PPO segments are hydrophobic and will attach to the surface of a hydrophobic membrane. The PEO segments are hydrophilic and will not attach to the membrane surface, but will extend into a hydrophilic environment. When PEO segments are attached to PPO segments, the resulting copolymer can be used to coat a polysulfone membrane surface and help repel or prevent molecules from contacting the surface of the membrane, thereby reducing the chance that those molecules will adsorb or deposit on the membrane surface. This helps to minimize the surface-induced thrombosis reaction that can occur on the surface of the polysulfone membrane during dialysis treatment.

The copolymer coating can be applied to the membrane by exposing the membrane surface to a solution of the copolymers dissolved in water. For example, a PEO—PPO—PEO copolymer can be dissolved in water to form the copolymer solution. Once the dialyzer has been assembled, the copolymer solution can be pumped through the dialyzer and the PEO—PPO—PEO triblock copolymers will attach to and coat the surface of the hydrophobic membrane. The copolymer solution can be pumped through only the blood compartment of a dialyzer so that the surface of the membrane facing the blood compartment is coated with the copolymers. The solution can also be pumped through both the blood compartment and the dialysate compartment of a dialyzer so that both sides of the membrane are coated with the copolymers.

### BRIEF DESCRIPTION OF THE DRAWINGS

FIG. 1 is a diagram of a patient undergoing dialysis treatment.

FIG. 2 is a diagram of a dialyzer and the inlet and outlet tubes connected to the dialyzer.

FIG. 2A is an enlarged cross-section diagram showing individual fibers in the dialyzer during treatment.

FIG. 3 is an enlarged diagram showing the diffusion of smaller molecules across a semipermeable membrane.

FIG. 4 is an enlarged cross-section diagram of the semipermeable membrane surface that has been coated on one side with a PEO—PPO—PEO triblock copolymer coating.

FIG. 5 shows a system used to single coat the semipermeable membrane in a dialyzer.

FIG. 6 shows a system used to double coat the semipermeable membrane in a dialyzer.

FIG. 7 is a graph showing the binding energy spectrum from the x-ray photoelectron spectroscopy of the surface of a coated polysulfone membrane.

FIG. 8 is a graph showing the summarized C1 peaks for an uncoated and coated polysulfone membrane.

FIG. 9 is a graph showing the summarized O1 peaks for an uncoated and coated polysulfone membrane.

FIGS. 10(A) and (B) are photographs from a scanning electron microscopy of a double coated and an uncoated polysulfone membrane at 5 kb×30 k.

FIGS. 11(A) and (B) are photographs from a scanning electron microscopy of a double coated and an uncoated polysulfone membrane at 5 kb×10 k.

FIGS. 12(A) and (B) are photographs from a scanning electron microscopy of a double coated and an uncoated polysulfone membrane at 5 kb×30 k.

FIG. 13 shows the chemistry of the synthesis of the Pluronic™ F 108 coating used in the labeled Pluronic™ F108 test.

FIG. 14 is a bar graph showing the average clotting time for samples of various membranes as compared to the negative control sample.

### DETAILED DESCRIPTION OF THE INVENTION

Hemodialysis, also called dialysis, is a medical procedure for patients with conditions such as renal failure where the patient's kidneys are no longer removing unwanted substances from the patient's blood. During dialysis, the patient's blood is cycled through a hemodialysis system so that unwanted substances are filtered from and/or wanted substances are added to the blood in a dialyzer. Patients who need this treatment may undergo the dialysis process several times each week so that these unwanted substances are regularly filtered from and/or wanted substances are regularly added to their blood. While this description discusses the invention in the context of dialysis, it will be appreciated by one skilled in the art that the invention can be used in a variety of medical procedures as well as other end stage renal disease treatments including but not limited to hemodialysis, hemoperfusion and hemodiafiltration.

FIG. 1 shows a patient 101 undergoing dialysis treatment with a hemodialysis system 102. The hemodialysis system 102 can be comprised of many different components and configured in a variety of ways. A patient is generally connected to the hemodialysis system 102 by an arterial line 103 and a venous line 104 during the treatment. The patient's blood is drawn from the patient 101 through the arterial line 103 by a pump 105 that is part of the hemodialysis system 102. The blood is pumped from the arterial line 103 into the dialyzer 106. The patient's blood flows through the dialyzer 106 where unwanted substances are filtered from or wanted substances are added to the blood. Once it has been filtered in the dialyzer 106, the patient's blood is returned to the patient 101 through the venous line 104 of the hemodialysis system 102.

The dialyzer acts as an “artificial kidney” and is used to filter unwanted substances from or add wanted substances to the patient's blood. The dialyzer is generally comprised of four basic parts: a casing, usually made of plastic; a blood compartment; a dialysate compartment; and one or more semipermeable membranes separating the blood compartment from the dialysate compartment. While dialyzers can be designed in many ways, the three most common designs for dialyzers have traditionally been the coil dialyzer, the parallel plate dialyzer and the hollow fiber dialyzer. Each of these designs work on the same principles, but the shape of the area or compartment for the patient's blood and the area or compartment for dialysate solution as well as the configuration of the semipermeable membrane are different. While this specification uses the hollow fiber dialyzer design to explain the invention disclosed herein, it can be appreciated by one skilled in the art that the invention could be used with any type of hydrophobic membrane, including membranes used in other types of dialyzers and other pieces of medical equipment.

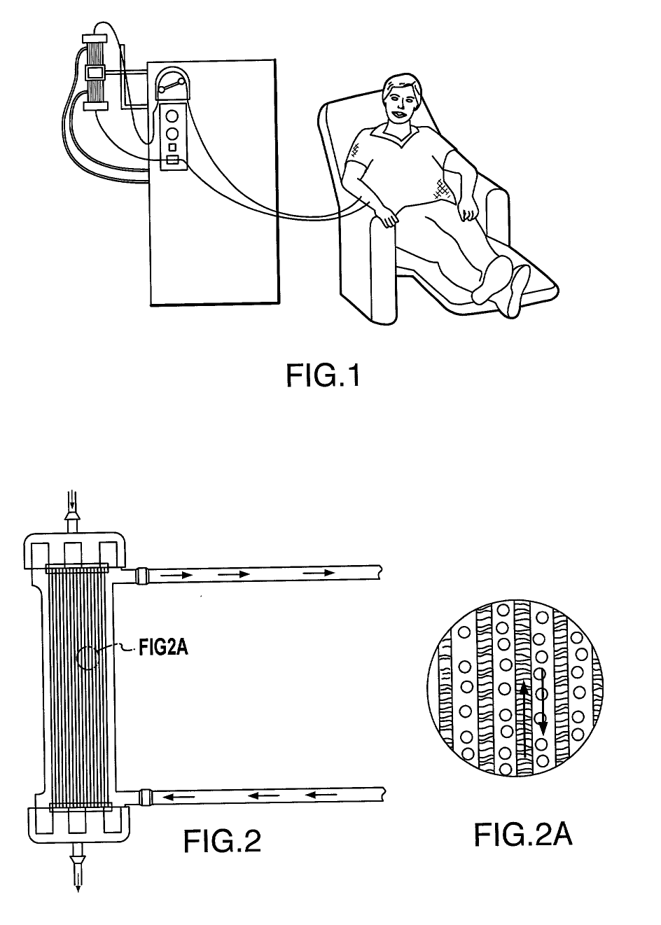

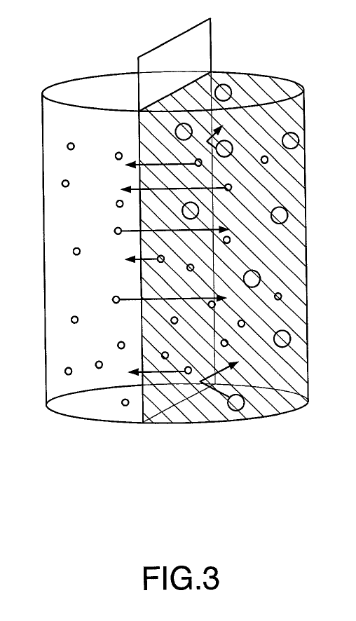

As shown in FIG. 2, the hollow fiber dialyzer 201 contains thousands of capillary-sized hollow fibers 202 made of a semipermeable membrane material that reach from one end of the dialyzer to the other end of the dialyzer. Each fiber may be as thin as a human hair with an internal diameter of approximately 150-300 microns. Fluid, such as the patient's blood, can flow through the hollow fibers 202. The fibers are usually held in place at each end of the dialyzer by a clay-like polyurethane “potting” material 203, which serves as the support structure for the hollow fibers 202. The hollow fibers 202 and structural support material 203 are enclosed in the plastic casing 204.

The patient's blood is pumped through the arterial line 210 and into an inlet chamber 211 at one end of the dialyzer 201. The blood then flows from the inlet chamber 211 through the hollow fibers 202, where it is filtered, and into an outlet chamber 213 at the other end of the dialyzer 201. The filtered blood is returned to the patient through the venous line 214. At the same time, the dialysate solution is pumped into the end of the dialyzer that contains the outlet chamber 213 through a dialysate inlet tube 216. The dialysate solution then flows in between the hollow fibers 202 to the end of the dialyzer that contains the inlet chamber 211 for the patient's blood. As shown in FIG. 2A, while the blood 218 inside the hollow fibers is flowing in the direction shown by arrow 220, the dialysate solution 219 can be flowing in between the hollow fibers in the direction shown by arrow 221. This creates a countercurrent flow as blood flows inside the hollow fibers in one direction (e.g., top to bottom in the diagram) and the dialysate flows around the hollow fibers in the opposite direction (e.g., bottom to top in the diagram). The dialysate solution then flows out of the dialyzer 201 through the dialysate outlet tube 217.

The semipermeable membrane has microscopic pores and generally divides the dialyzer into two separate areas or compartments: one area for the patient's blood and one area for the dialysate solution. The patient's blood is on one side of the semipermeable membrane and the dialysate solution is on the other side of the membrane. The semipermeable membrane is not necessarily one contiguous membrane, but may be comprised of several separate membranes that generally divide the dialyzer into two distinct areas. For example, in the hollow fiber dialyzer, each hollow fiber is made of the semipermeable membrane material.

Unwanted substances are removed from and/or wanted substances are added to the patient's blood by flowing through the pores in the semipermeable membrane between the blood and dialysate solution. The substances flow through the pores in the membrane as a result of diffusion and/or pressure differentials between the blood 218 and dialysate solution 219 on either side of the membrane.

Under the principle of diffusion, substances will move, where possible, from an area of greater concentration to an area of lesser concentration. During dialysis, substances or molecules in a patient's blood that are small enough to move through the microscopic pores in the dialyzer's semipermeable membrane will pass through the membrane from the patient's blood to the dialysate solution if the concentration of those substances or molecules is greater in the patient's blood than in the dialysate solution. In other words, the filtered substances will move from the area of greater concentration in the patient's blood to the area of lesser concentration in the dialysate solution, through the pores in the semipermeable membrane.

Similarly, substances or molecules in the dialysate solution that are small enough to move through the microscopic pores in the dialyzer's semipermeable membrane will also pass through the membrane from the dialysate solution to the patient's blood if the concentration of those substances or molecules is greater in the dialysate solution than in the patient's blood. Consequently, the diffusion of a substance between the blood and the dialysate solution will be controlled by at least two factors: (i) the relative difference in concentration of that substance in the dialysate solution as compared to the concentration of that substance in the patient's blood (i.e., the concentration gradient), and (ii) the size of the microscope pores in the semipermeable membrane of the dialyzer.

In addition, substances can flow across the membrane as a result of the pressure differentials between the blood and dialysate solution. Substances will tend to flow from areas of higher pressure to areas of lower pressure. Therefore, by controlling the relative pressure differentials between the blood and dialysate solution, substances may tend to flow either from the blood to the dialysate solution, or from the dialysate solution to the blood.

The dialysate solution acts as a buffer solution and is typically an electrolyte solution that contains substances such as purified water, sodium, potassium, calcium, magnesium, chloride and dextrose. The dialysate solution helps to regulate the flow of substances and molecules through the membrane by creating the concentration gradient. The concentration of the substances or molecules in the dialysate solution can be adjusted to increase or decrease the gradient for those substances that need to be filtered from or added to the patient's blood. If a substance has molecules small enough to pass through the pores of the semipermeable membrane, the concentration gradient between the blood and the dialysate solution can cause that substance to move from the patient's blood to the dialysate solution if the concentration of that substance is greater in the patient's blood. Conversely, the concentration gradient can cause a substance to move from the dialysate solution to the blood if the concentration of that substance is greater in the dialysate solution. Therefore, the concentration of substances in the dialysate solution can be adjusted to target the removal or addition of certain substances from the patient's blood.

The semipermeable membrane has microscopic pores that allow only certain sized molecules, such as specific solutes, electrolytes and water, to pass through the membrane. The size of these pores prevents larger molecules in the blood, such as medium sized proteins or red blood cells, from diffusing across the membrane into the dialysate solution.

As shown in FIG. 3, the patient's blood 301 is separated from the dialysate solution 302 by the semipermeable membrane 303. Substances or molecules that are too large to pass through the pores of the semipermeable membrane are unable to diffuse across the membrane 303 and remain in the patient's blood or dialysate solution. Substances and molecules that are small enough to pass through the pores in the semipermeable membrane 303 can diffuse between the patient's blood 301 and the dialysate solution 302 depending upon the relative concentration of those substances in the blood 301 and the dialysate solution 302.

Plasma proteins in the blood can adsorb or deposit on the surface of the polysulfone membrane which can result in a membrane surface-induced thrombosis reaction. The plasma proteins are larger molecules that cannot pass through the microscopic pores of the semipermeable membrane, but can adsorb or deposit on the surface of the membrane. Depending upon the surface of the semipermeable membrane in the dialyzer, the total amount of plasma protein found on the membrane surface after filtration has been as low as 400 mg/m² or as high as 2,600 mg/m². The range of total plasma protein found on the membrane surface depends, at least in part, on the membrane material used in the dialyzer. Therefore, it is important to use membrane materials that will help reduce and minimize protein adsorption and deposition on the surface of the membrane during the dialysis treatment.

The properties of the membrane surface can be altered with surface modification techniques to help minimize the risk of protein adsorption. PEO and polyethylene glycol (PEG) are biocompatible materials that can be used to modify the surface of hydrophobic membranes. PEO and PEG can be attached to other substances that can be immobilized onto the surface of a polysulfone membrane by processes such as wet chemical reaction, plasma grafting, electron beam irradiation or physical adsorption methods. PEO chains are hydrophilic so that they will not attach to a hydrophobic membrane surface, but they will extend into a hydrophilic environment such as the patient's blood. When these PEO chains are attached to a hydrophobic material such as PPO, the resulting copolymer can be used to coat a hydrophobic membrane surface. These copolymers can be used to minimize the surface-induced thrombosis reaction that can occur on the surface of a polysulfone membrane during dialysis treatment. While these copolymers have been found to work particularly well for coating the surfaces of membranes used in dialysis treatment, it will be appreciated by one skilled in the art that these copolymers can be used to coat the surfaces of membranes for other purposes as well.

There are many different kinds of copolymers that have a hydrophobic segment and hydrophilic segment and can be used to coat hydrophobic membrane surfaces. One group of copolymers that has been found to work particularly well for coating membrane surfaces are PEO—PPO—PEO triblock copolymers. These copolymers are commercially available from BASF and sold under the trademark name of Pluronics™. These copolymers have been used to modify the surface of hydrophobic polystyrene.

The PEO—PPO—PEO triblock copolymers work well because the hydrophobic PPO segment attaches to the surface of the hydrophobic membrane and the hydrophilic PEO segments on either side of the PPO segment extend from the membrane surface into the patient's blood, helping to repel larger molecules in the patient's blood and prevent them from adsorbing or depositing on the surface of the membrane. The PEO segments can be attached to both ends of a PPO segment.

The PPO segment will adsorb on hydrophobic substrates, such as polystyrene or polysulfone, by hydrophobic interactions. When the hydrophobic PPO segment has adsorbed onto the polysulfone membrane surface, the hydrophilic PEO segments extend from the PPO segment into the hydrophilic environment and help repel substances and molecules from the surface of the membrane, preventing the substances and molecules from contacting the surface of the polysulfone membrane. After the PEO—PPO—PEO triblock copolymers have coated the membrane surface, the hydrophobic surface of the polysulfone membrane is essentially turned into a hydrophilic surface. While smaller molecules can flow past the PEO segments of the PEO—PPO—PEO triblock copolymers and pass through the microscopic pores in the semipermeable membranes, larger molecules which cannot flow through the pores in the membrane are repelled by the PEO segments of the PEO—PPO—PEO triblock copolymers and prevented from adsorbing on the membrane surface. This helps reduce the chance of a surface-induced thrombosis reaction that can trigger blood coagulation and endanger the patient.

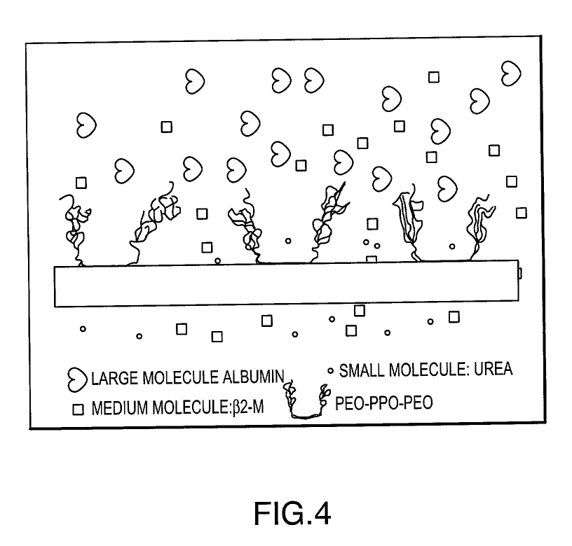

As shown in FIG. 4, the membrane 401 can be coated with PEO—PPO—PEO triblock copolymers 402. Although not shown in this FIG., the membrane 401 is porous so that certain sized molecules and substances can pass through the pores in the membrane 401. Only the top side of the membrane, which faces the blood compartment in the dialyzer, has been coated in this FIG. When the PEO—PPO—PEO triblock copolymers coat the membrane, the PPO segment 411 of the PEO—PPO—PEO triblock copolymer 402 adsorbs on the membrane surface. The PEO segments 411 which are attached on either end of the PPO segment 410 do not adsorb to the membrane surface 401, but extend into the hydrophilic environment.

Once the membrane surface has been coated, the copolymers will help prevent larger molecules in the blood from contacting the membrane surface. Larger molecules such as albumin 403 are unable to pass through the microscopic pores in the semipermeable membrane 401. Therefore, they cannot pass through the membrane from the blood into the dialysate. In contrast, medium sized molecules such as β₂-microglobulin 404 and smaller molecules such as urea 405 can pass through the microscopic pores in the semipermeable membrane 401 and pass from one side of the membrane to the other. The copolymer coating helps prevent larger molecules from contacting the membrane surface and increasing the risk of blood coagulation or other dangers to the patient undergoing dialysis treatment, but does not prevent smaller molecules from passing through the pores in the membrane.

Other substances or molecules can be targeted for removal from a patient's blood by immobilizing ligands on the copolymers used to coat the membrane. The immobilized ligands will extract specific targets, such as protein, from the patient's blood because the targets will bind to the immobilized ligands.

To immobilize ligands on the surface of the membrane, an active group can be added to the copolymer used for coating the membrane surface. For a PPO—PEO—PPO triblock copolymer, the active group can be attached to the end of the PEO segment that is not attached to the PPO segment. The active group will bind to certain ligands, thereby immobilizing the ligands on the copolymer. For example, a modified PEO—PPO—PEO triblock copolymer where at least one of the PEO segments has an organic metal-chelating end group (R) attached to its end is disclosed in U.S. Pat. No. 6,087,452. This type of modified surfactant can also be used to coat the surface of a hydrophobic membrane. The ligands are immobilized on the surfactant through the metal-chelating end group.

Other types of active groups can also be used to immobilize ligands on the copolymers so that the ligands can bind with targets. U.S. Pat. No. 5,516,703 discloses other examples of active groups, such as the primary amine NH₂ group or pyridyl disulfide group. When used to coat the surface of dialyzer membranes, these other active groups and ligands can also be used to remove specific targets from the patient's blood.

The modified copolymer with the ligands immobilized by an active group serves the dual purpose of repelling biological molecules, such as protein, from adsorbing or depositing on the membrane surface and extracting from the patient's blood specific targets, such as protein, as they bind to the immobilized ligands. During dialysis, the copolymer coating with an active group can immobilize ligands, such as antibodies, that will bind with targets, such as protein, thereby extracting that specific target from the patient's blood.

The membrane surface can be coated with copolymers in many different ways. However, since a hydrophobic segment, such as PPO, can adsorb on the hydrophobic surface of a polysulfone membrane by hydrophobic interactions, a copolymer with a hydrophobic segment will adsorb to the polysulfone membrane surface when a solution containing the copolymer is exposed to the surface of the polysulfone membrane for a period of time. This has been found to work particularly well with the PEO—PPO—PEO triblock copolymer.

This method can be used to coat a hydrophobic membrane in a dialyzer. A solution containing the triblock copolymer can be pumped through the dialyzer after the dialyzer has been assembled. The membrane in the dialyzer will be exposed to the solution as the solution is pumped through the dialyzer and the triblock copolymer will adhere to and coat the hydrophobic membrane surface.

Commercially available PEO—PPO—PEO triblock copolymers are a powdered substance that can be dissolved in water to create a solution that can be used to coat a membrane. For example, the following three Pluronics™ are commercially available from BASF and have been used to coat a polysulfone membrane in a dialyzer: Pluronic™ F68 [(PEO)₇₆—(PPO)₃₀—(PEO)₇₆]; Pluronic™ F88 [(PEO)₁₀₄—(PPO)₃₉—(PEO)₁₀₄]; and Pluronic™ F108 [(PEO)₁₂₉—(PPO)₅₆—(PEO)₁₂₉]. The chemical structure formula for these three Pluronic surfactants is:

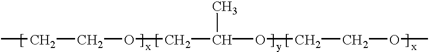

| Pluronic Surfactant | X | Y | Molecular Weight |
|---|---|---|---|
| F68 | 76 | 30 | 8,400 |
| F88 | 104 | 39 | 11,400 |
| F108 | 129 | 56 | 14,600 |

Other copolymers with at least one hydrophobic segment and at least one hydrophilic segment can be used as well.

The PEO-PPO-PEO triblock copolymer solution can be pumped through the dialyzer. The hydrophobic segment of the triblock copolymer will adhere to the surface of the hydrophobic membrane, thereby coating it as the solution contacts the membrane surface.

This coating process has been found to work particularly well when the PEO—PPO—PEO triblock copolymers are dissolved in reverse osmosis deionized (RO DI) water to form an approximately 0.2% (weight/volume) solution. While the coating process will work when the weight/volume of the solution is greater or less than 0.2%, the weight/volume of the solution should be less than the critical gel point for the solution so that the solution can easily flow through a dialyzer. The critical gel point for a solution of Pluronic™ F108 is approximately 3% (weight/volume).

During the coating process, the solution must be maintained at a temperature above freezing, and it is believed that the coating process will work well when the solution has a temperature greater than 20° C. This coating process for a dialyzer membrane has been found to work particularly well when the solution is maintained at a temperature of about 37° C., which is the normal temperature for human blood.

While the solution can be pumped through the dialyzer at a higher or lower flow rate, the coating process has been found to work particularly well when the solution is pumped through the dialyzer at a flow rate of approximately 300 ml/minute. While the solution can be pumped through the dialyzer for more or less time, it has been found that pumping the approximately 0.2% (weight/volume) solution through the dialyzer at a flow rate of approximately 300 ml/minute for approximately 30 minutes is a sufficient amount of time for the PEO—PPO—PEO triblock copolymers in the solution to coat the surface of the polysulfone membrane. It will be appreciated by one skilled in the art that the coating process does not have to take place after a membrane has been assembled in a dialyzer. For example, a hydrophobic membrane could be coated with a copolymer by immersing the membrane in a reservoir of the copolymer solution.

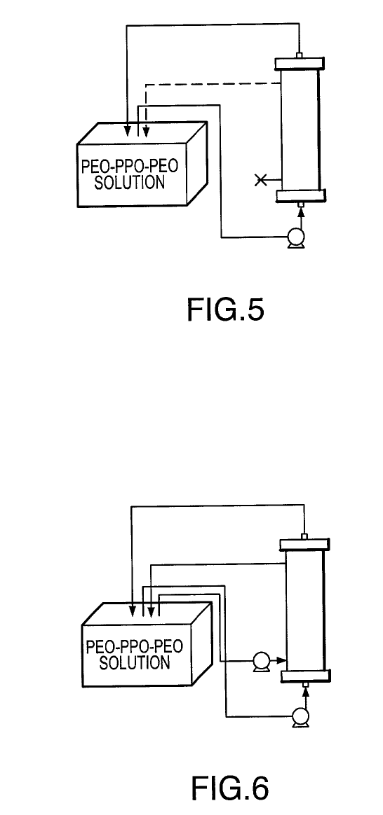

As shown in FIG. 5, the membrane can be coated with a single coating process whereby the copolymer solution is pumped through only the blood compartment of the dialyzer. During the single coating process, one pump 502 is used to pump the solution from a reservoir of solution 501 into the outlet chamber 503 of the dialyzer 507. When the outlet chamber 503 fills with the solution, the solution flows up through the hollow fibers in the dialyzer 507 and into the inlet chamber 504 of the dialyzer 507. When the inlet chamber 504 is filled with the solution, the solution flows out of the inlet chamber 504 and can be returned to the reservoir of solution 501 to be reused in the coating process. The dialysate inlet tube 505 is plugged so that any part of the copolymer solution that passes through the membrane cannot leave the dialyzer through the dialysate inlet tube 505. Therefore, the dialysate compartment may also fill up with the solution to the extent the solution passes through the semipermeable membrane during the coating process. If the dialysate compartment fills up with solution during the single coating process, the solution can be returned to the reservoir of solution 501 through the dialysate outlet tube 506. Alternatively, the dialysis outlet tube 506 can be plugged so that the solution can only exit the dialyzer 507 through the inlet chamber 504 of the dialyzer 507.

As shown in FIG. 6, the membrane can also be coated with a double-coating process whereby the copolymer solution is pumped through both the blood compartment and the dialysate compartment of the dialyzer. During the double-coating process, two pumps 602 are used to pump the solution from the reservoir of solution 601 through the dialyzer 607. The solution is pumped from the reservoir 601 into both the outlet chamber 603 and the dialysate inlet tube 605. When the outlet chamber 603 fills with the solution, the solution flows up through the hollow fibers in the dialyzer 607 and into the inlet chamber 604 of the dialyzer 607. When the inlet chamber 604 is filled with the solution, the solution once again flows out of the inlet chamber 604 and can be returned to the reservoir of solution 601 to be reused in the coating process.

At the same time, the dialysate chamber of the dialyzer 607 is being filled with the solution and the solution flows up around the hollow fiber of the dialyzer 607, filling the dialysate chamber of the dialyzer 607. When the dialysate compartment is filled with the solution, the solution flows out of the dialysate outlet tube 606 and can be returned to the reservoir of solution 601 to be reused in the coating process. By using this double coating process, both sides of the membrane are directly exposed to the copolymers in the solution and are therefore coated.

One major advantage to using this process to coat a membrane for a dialyzer over other methods, such as coating the membrane before the dialyzer is assembled, is that the membrane coating process can take place after the dialyzer has been assembled. Therefore, additional steps would not have to be added to alter or change the existing dialyzer production process. Furthermore, the membrane is coated after it is in place in the dialyzer making it less likely that the coating will be disrupted before the dialyzer is used in treatment.

### Experimental Results

The results from two tests indicate that the PEO—PPO—PEO triblock copolymer is an effective coating for polysulfone membranes used in dialyzers and can reduce the chance of thrombosis reaction during dialysis treatment.

### Test 1. Surface Characteristics

A test was conducted in which the surfaces of a coated polysulfone membrane in a dialyzer and an uncoated polysulfone membrane in a dialyzer were examined under high powered microscopes to see whether the coating appeared to alter the characteristics of the membrane surface. The surface of the coated polysulfone membrane was coated with Pluronic™ F108 using the double coating process described above. A solution with approximately 0.2% (weight/volume) of the F108 was pumped through both the blood compartment and the dialysate compartment at approximately 300 ml/minute for approximately 30 minutes.

The surface characterization of the coated membrane and the uncoated membrane were then examined by three methods: X-ray photoelectron spectroscopy (XPS), scanning electron microscopy (SEM) and a labeled Pluronic™ surfactant. The results from the examination of the coated membrane were compared with the results of the examination of the uncoated membrane to determine whether the coating affected various surface characteristics of the membrane.

### 1. XPS

XPS, also known as Electron Spectroscopy for Chemical Analysis, is generally used to analyze the surface characterization of biomedical polymers. This analysis provides a total elemental analysis of the top 10˜200 Å of the membrane surface, thereby revealing what elements comprise the membrane surface.

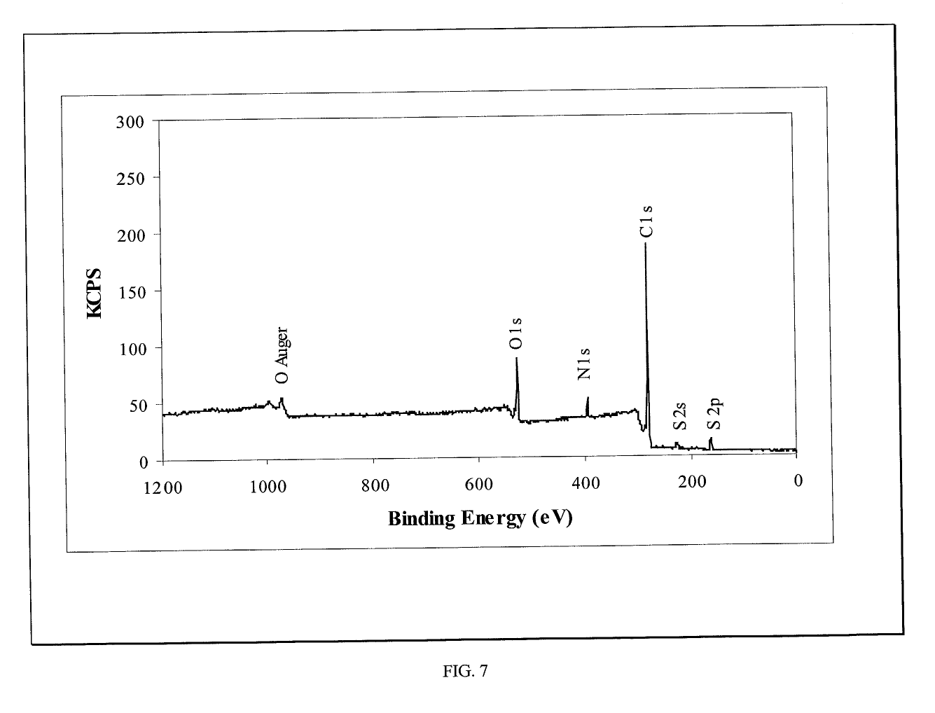

An XPS survey scan (wide scan, 0˜1000 eV, full range of the elemental analysis) was used to analyze the surface of the uncoated polysulfone membrane and the coated polysulfone membrane. FIG. 7 shows that all four major elements, sulfur, carbon, oxygen and nitrogen, were present on the surface of a polysulfone fiber from the coated membrane surface. The carbon, oxygen and sulfur were expected because they are part of the chemical structure of the polysulfone. However, the reasons for the presence of nitrogen on the surface of the polysulfone fiber is not readily apparent because there is no nitrogen in the chemical structure of polysulfone or the PEO—PPO—PEO triblock copolymer used to coat the membrane. The presence of nitrogen on the polysulfone fiber is probably left from the use of polyvinyl pyrrolidone (PVP) in the process to make the polysulfone fibers. Since the XPS survey scan shows the presence of nitrogen on the membrane surface, this indicates that the PVP was present on the surface of the polysulfone membrane, indicating that the PVP adhered to the membrane surface.

An XPS high-resolution scan (narrow scan, only 20 eV for each element) was used to analyze both the uncoated polysulfone membrane and the coated polysulfone membrane. This scan results in a detailed chemical structure of each membrane surface. This scan showed significant differences between the C and O peaks for the coated and uncoated membrane due to the chemical structure of the F108 triblock copolymer used to coat the membrane surface. However, neither the S nor the N peaks showed significant differences between the two membranes.

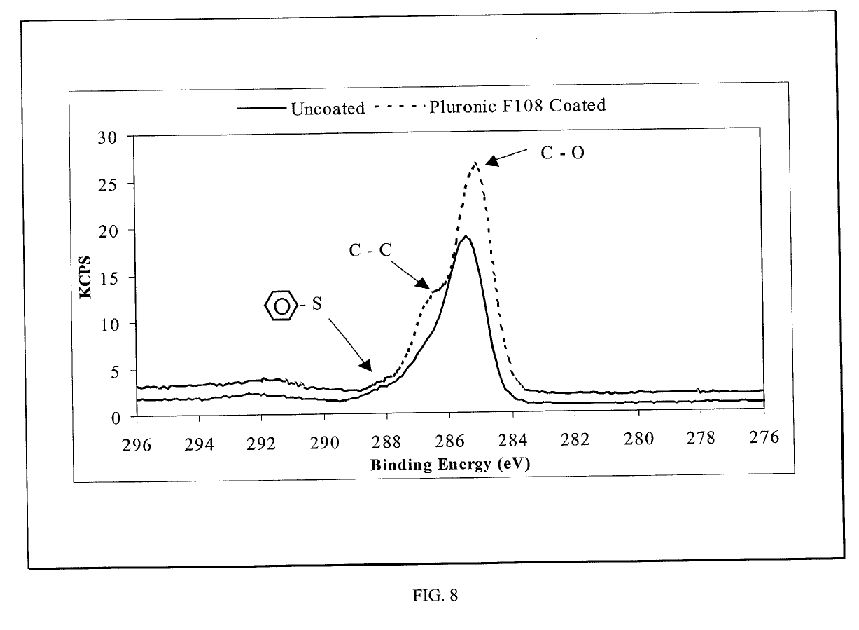

The summarized C1s peaks for the uncoated membrane and the coated membrane are shown in FIG. 8. Each C1s peak contains three different sub-peaks that represent different chemical bonds:

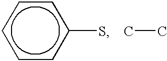

C—C and C—O. The C—O peak for the coated polysulfone fiber (dashed line) was greater than the peak for the uncoated polysulfone fiber (solid line) and shifted to a lower binding energy. This peak indicates that the F108 copolymer adhered and bound to the polysulfone fiber surface.

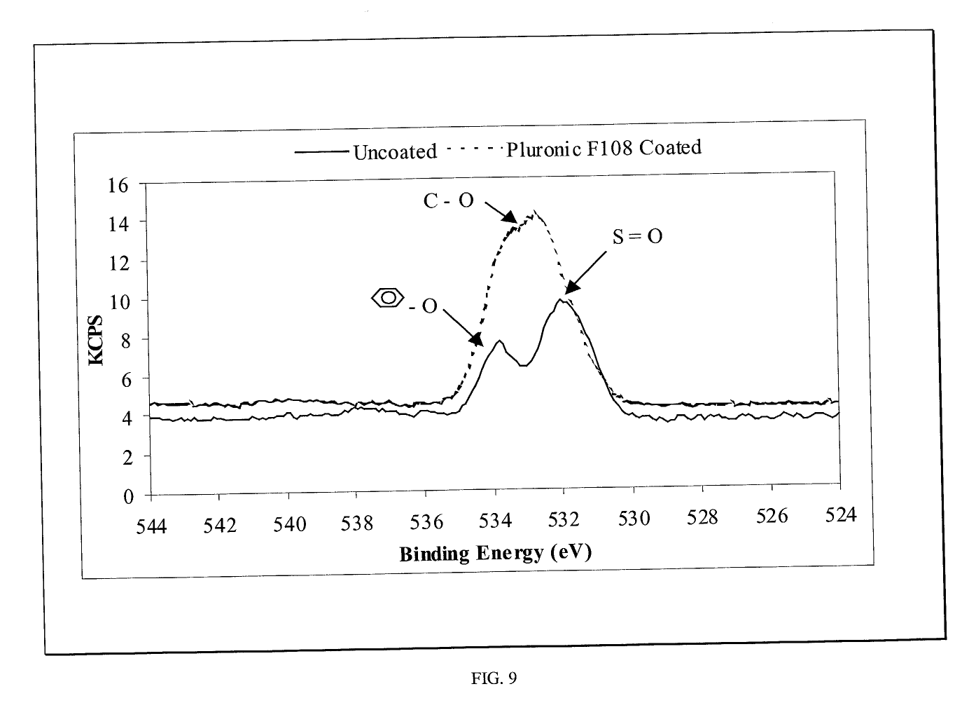

As shown in FIG. 9, the O1s peak for the uncoated polysulfone fiber is also significantly different than the peak for the coated polysulfone fiber. Each O1s peak contains three sub-peaks representing three different chemical bonds:

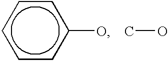

C—O and S═O. The binding energy of C—O and S═O for both the coated and uncoated fiber are similar and it is difficult to separate these two peaks. Therefore, only two sub-peaks are shown for the coated membrane.

The XPS spectrum shows the chemical analysis for the surface of the fibers. A summary of the chemical analysis in this study is listed in Table 1. The most significant change in atomic percentages between the uncoated and the coated polysulfone fibers was for the oxygen atom. Compared to the oxygen content of the uncoated polysulfone fiber, the oxygen content of the coated polysulfone fiber increased 3% (form 12.5% to 15.5%). Most of this oxygen should have come from the F108 coating. The carbon content did not change much due to the large amount of the carbon atom also found in the F108. On the other hand, the nitrogen content was decreased 4.4% (from 6.7% to 2.3%) because there was no additional nitrogen on the polysulfone fiber surface after the polysulfone fiber was coated with the F108.

TABLE 1

Atom Percentage (%)

| Sample | O | N | C | S |
|---|---|---|---|---|
| Uncoated Polysulfone | 12.5 | 6.7 | 78.3 | 2.5 |
| Pluronic ™ F108 | 15.5 | 2.3 | 79.1 | 3.1 |
| Coated Polysulfone | | | | |

The XPS results show the presence of the F108 coating on the coated polysulfone fibers, indicating that the coating process worked.

### 2. SEM

The SEM microscope produces high resolution images, which allow closely spaced features of an object to be examined at a high magnification. In this case, the SEM microscope was used to compare the inside and outside surface of the uncoated polysulfone membrane and the coated polysulfone membrane. Both polysulfone fibers were coated with a thin layer of platinum under a vacuum for 90 seconds (approximately 1 nm) to make the fibers conductive so that they could be examined using this process.

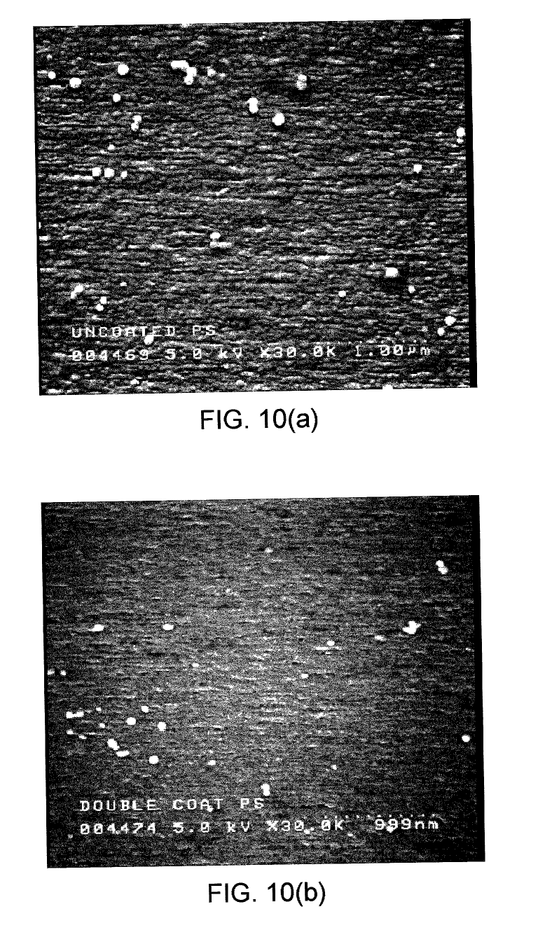

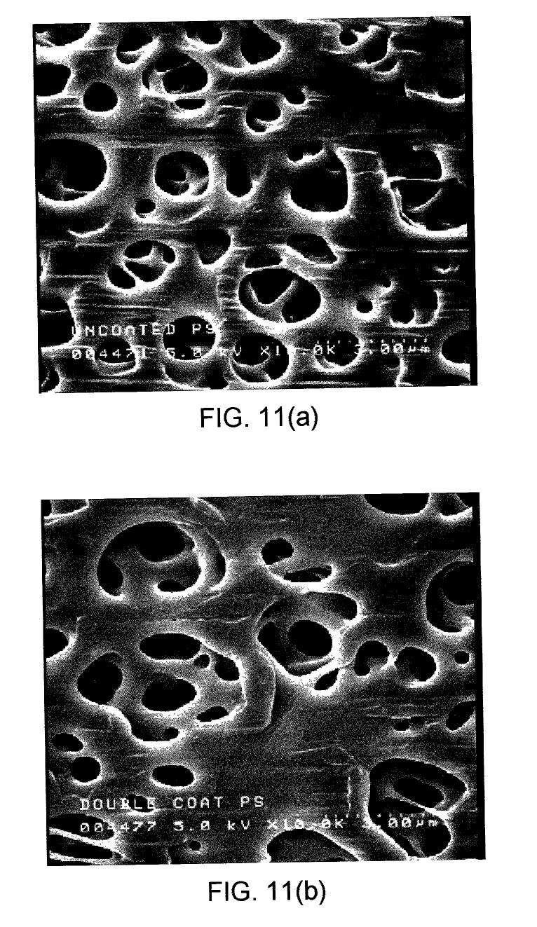

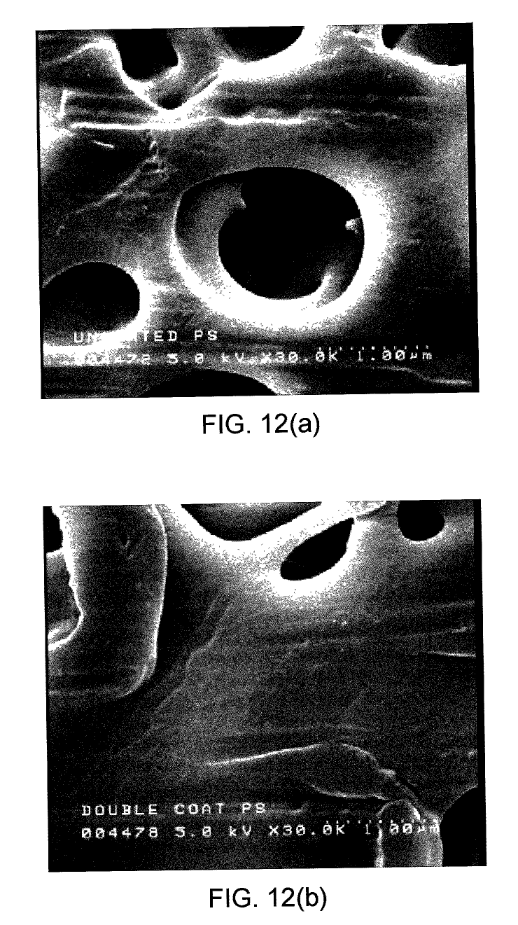

The purpose of the SEM analysis is to determine if the coating alters the pore size or blocks the porous surface of the polysulfone membrane. The SEM images for the uncoated and coated polysulfone fibers are shown in FIGS. 10-12. In each FIG., image (a) is the uncoated polysulfone membrane and image (b) is the double coated polysulfone membrane. FIG. 10 contains SEM images for the inside surface of the polysulfone membranes at 5 kb×30 k. FIG. 11 contains SEM images for the outside surface of the polysulfone membranes at 5 kb×10 k. FIG. 12 contains SEM images for the outside surface of the polysulfone membranes at 5 kb×30 k.

Although the pore distribution for images (a) and (b) in FIG. 11 are slightly different, the overall pore distribution of the outside surface of the uncoated membrane is similar to the coated membrane. FIGS. 12(a) and (b) are used to determine whether the coating is on the surface of the non-porous regions of the coated membrane. An analysis of FIGS. 12(a) and (b) indicate no significant difference. In fact, all the SEM images show no significant difference between the uncoated membrane and the coated membrane. This indicates that the F108 coating did not block the pores of the polysulfone fiber membrane.

### 3. Labeled Pluronic™ F108

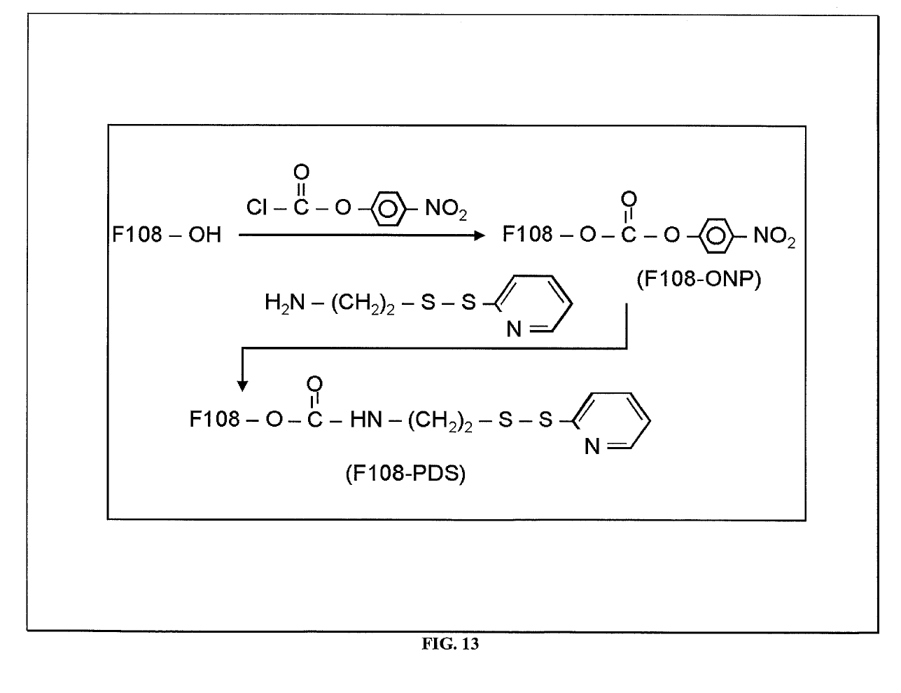

The labeled Pluronic™ F108 test quantitatively determines the amount of F108 coating on the dialyzer surface in terms of the surface coverage of the F108. The labeled Pluronic™ F108 has probe molecules covalently bound to the F108. This probe molecule can be a radioisotope, fluorescent or colorimetric molecule. The pyridyl disulfide (“PDS”) group covalently bound to the PEO end of the F108 molecule was selected in this study because of its stability in the aqueous solution. The chemistry of the synthesis of F108-PDS is shown in FIG. 13 and the detailed procedure for this portion of the test can be found in the following publication: J -T. Li, J. Carlsson, J -N Lin and K. D. Caldwell, “Chemical Modification of Surface Active Poly (ethylene oxide)-Poly (propylene oxide) Triblock Copolymers”, Bioconjugate Chem., vol. 7, (1996), p. 592-99. In this synthesis process, F108 was first modified with 4-nitrophenyl chloroformate in the benzene. This reaction converts the —OH group of F108 into a p-nitrophenyl (—ONP) group, which is easily reacted with the primary amine group. The product of this reaction is F108-ONP. The F108-ONP then reacts with 2-(2-pyridyldithio)ethylamine (NH₂—PDS). The primary amine group of the 2-(2-pyridyldithio)ethylamine will react with the ONP group of F108-ONP in the methanol. The product of this reaction is the F108-PDS.

The experimental procedure used was as follows: a liter of 0.2% F108-PDS solution was prepared for the dialyzer coating process. The single coating process was used and 0.2% F108-PDS solution was pumped through the blood side of a F80A dialyzer at 37° C. for 30 minutes. At the end of the single coating process, the F108-PDS solution flowed back to the reservoir. A sample of the F108-PDS before and after the coating process was collected from the solution reservoir to determine the amount of F108 that was depleted from the solution during the coating process.

RO DI water was then pumped through the blood compartment of the dialyzer for another 30 minutes to wash out any F108-PDS that did not attach to the surface of the membrane. The excess water was removed from the dialyzer by forcing air through the blood side of the dialyzer. A liter of 2.5 mM dithiothreitol (DTT) solution was then circulated through the dialyzer for 30 minutes, and a 2 ml sample was taken from the DTT reservoir. All the samples were measured using UV absorbance at 343 nm to determine the quantity of F108.

Prior to the analysis of F108-PDS, the F108-PDS was characterized for the degree of labeling (i.e., how many PDS molecules covalently bound to each F108 molecule). A 2 ml F108-PDS solution with the concentration of 2 mg/ml was prepared in RO DI water. A 0.2 ml of 25 mM DTT solution was added into this F108-PDS solution and the mixture was allowed to react for 10 minutes at room temperature. The DTT will break the disulfide bound of PDS and release the 2-thiopyridone molecules into the solution. The UV absorbance (A) of this solution at wavelength of 343 nm was measured. The concentration of released 2-thiopyridone ([PDS]) was calculated using a molar extinction coefficient (ε) of 8060 cm⁻¹ M⁻¹ in Eq. (1):

[PDS]=A/ε Eq. (1)

The degree of labeling (DL) then was calculated using Eq. (2)

DL=[PDS]/[F108-PDS] Eq. (2)

where

[F108-PDS] is the concentration of the F108-PDS, which is 2 mg/ml in this study. The calculated DL in this study was 0.83.

The result showed that 0.35 g of F108-PDS was coated on the dialyzer. Comparing the F108-PDS solution prior to and after the coating process, 0.05 g of F108-PDS was lost during the washing step. Based on the published size of each F108 molecule as 7.9 nm², the total coated area of the F108 on the F80A dialyzer was calculated as 114 m². The surface coverage (SC) was calculated as the surface area of F108 divided by the surface area of the membrane in the F80A dialyzer. The calculated SC was 63, which is far more than a monolayer of F108 on the polysulfone surface. F80A dialyzers have a surface area of 1.8 m². This estimates only the inner surface of the F80A dialyzer and does not count the surface area of the membrane pores. Since the average molecular weight of F108 is 14,600 Da, which is similar to the size of lysozyme, the F108 molecule could penetrate into the membrane wall. This result indicates that the F108 not only covers the inner surface of the polysulfone membrane, but also some of the surface area of the pores inside the wall of the polysulfone fibers.

### Test 2: Impact on Blood Coagulation Time

The partial thromboplastin time test (PTT) is used to determine the amount of time it takes for blood to coagulate when exposed to various substances or surfaces. The PTT is used as a general screening test for the detection of coagulation abnormalities in the intrinsic pathway. The PTT was used to test the effectiveness of the Pluronic™ F108 coating on the surface of a polysulfone membrane used in a dialyzer.

The polysulfone membranes from six dialyzers were used in this test: (1) uncoated polysulfone; (2) Pluronic™ F108 [(PEO)129—(PPO)₅₆—(PEO)₁₂₉] (purchased from BASF) coated polysulfone; (3) Acrylonitrile-Sodium Methally Sulfone; (4) Cellulose; (5) Polyethersulfone; and (6) PEO coated Cellulose. In addition, the study also included two additional samples that were tested under the same conditions: a negative control sample that included only plasma and no membrane material, and a positive control sample that contained glass beads instead of a membrane material. The dialyzer membranes used in this test were coated with the single coating process described above and sent to the Nelson Laboratories in Salt Lake City, Utah which performed the PTT test in accordance with the Nelson Laboratories protocol NO200013003-01.

First, 120 mg of the membrane was placed in a test tube containing 200 ul human plasma and incubated at 22° C. for 60 minutes. The human plasma contained 0.01 M of sodium citrate. Next, the test tube containing the prepared PTT reagents and 0.02 M CaCl₂ was placed in a 37° C. water bath to increase the test tube temperature to 37° C. Then, 200 ul PTT reagent was transferred into one sample test tube and incubated at 37° C. for exactly three minutes. 200 ul of 0.02 M CaCl₂ solution at a temperature of 37° C. was added to the test tube and mixed well. The time it took for a blood clot to form in the test tube was measured and recorded.

Six test tubes for each sample (including the negative and positive control samples) were measured in this study. Results from this test show that the membrane coated with the F108 did improve the hemocompatibility of the polysulfone membrane.

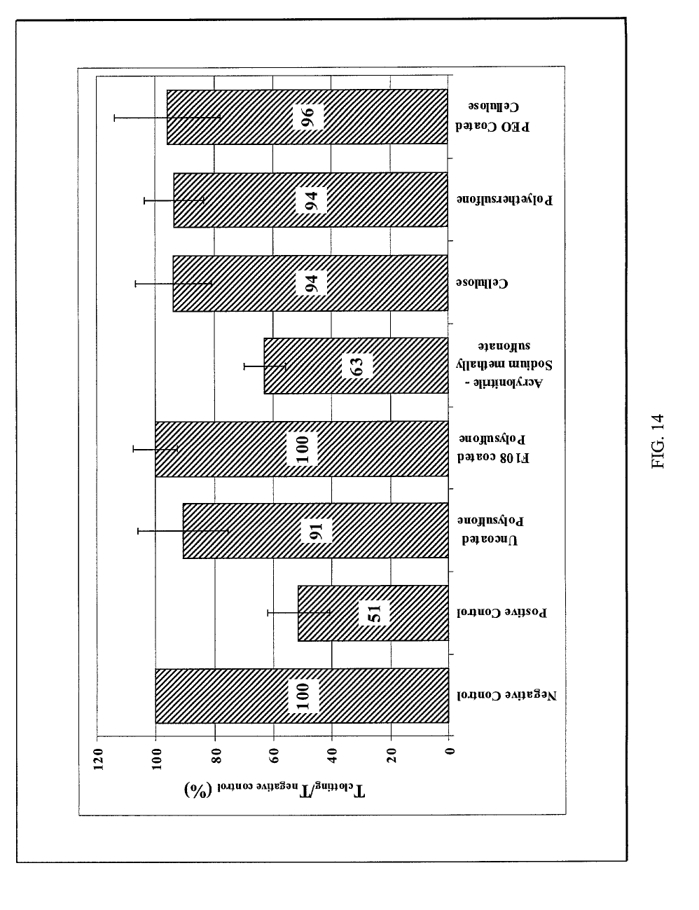

FIG. 14 shows the average clotting time for each of the 8 samples tested. The extensions on the top of the bar graph for each of the 7 samples (other than the negative control sample) show the magnitude of error in the average clot time.

Table 2 shows the clotting time ratio for each of the seven samples as compared to the negative control sample. The clotting time ratio is equal to the plasma clotting time of the sample divided by the plasma clotting time of the negative control.

Clotting Time=Tₛₐₘₚₗₑ/T_{negative control} Eq. (3).

The plasma clotting time for the negative control is 100%. The higher percentage of the clotting time ratio indicates that the plasma needed a longer time to clot and that the material is, therefore, more hemocompatible.

TABLE 2

| Membranes | Clotting Time Ration (%) |
|---|---|
| Positive Control | 51 ± 10.64 |
| Uncoated Polysulfone | 91 ± 15.40 |
| Pluronic ™ F108 Coated Polysulfone | 100 ± 7.58 |
| Acrylonitrile - Sodium Methally Sulfonate | 63 ± 7.01 |
| Cellulose | 94 ± 12.88 |
| Polyethersulfone | 94 ± 10.15 |
| PEO Coated Cellulose | 96 ± 18.11 |

These results indicate that the F108 coating on the polysulfone membrane had almost no effect on the plasma clot formation time. In contrast, the average plasma clot formation time for each of the other substances was 96% or lower, indicating that contact with the surface of these substances tends to decrease the average plasma clot formation time and increase the risk of clotting. Consequently, this test indicates that the F108 coating improves the hemocompatibility of the polysulfone membrane.

We claim:

1. A coated membrane for use in dialysis, hemodialysis, hemofiltration, hemodiafiltration or other end stage renal disease treatments comprising a hydrophobic membrane wherein at least one copolymer is attached to said hydrophobic membrane, and each copolymer is comprised of at least one hydrophobic segment and at least one hydrophilic segment.

2. A coated membrane as in claim 1, wherein said hydrophobic membrane is a semipermeable polysulfone membrane or semipermeable polyethersulfone membrane.

3. A coated membrane as in claim 1, wherein said hydrophobic segment is PPO and said hydrophilic segment is PEO.

4. A coated membrane as in claim 1, wherein said copolymer has the chemical structure formula:

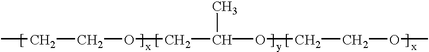

5. A coated membrane as in claim 4, wherein the x=129 and the y=56 in said formula.

6. A coated membrane as in claim 1, wherein at least one of said hydrophilic segments has a modified active group attached to it.

7. A coated membrane as in claim 6, wherein said modified active group is comprised of at least one of a metal-chelating end group, primary amine group or pyridyl disulfide group.

8. A dialyzer for use in dialysis, hemodialysis, hemofiltration, hemodiafiltration or other end stage renal disease treatments having a hydrophobic membrane with at least one copolymer attached to said hydrophobic membrane, wherein said copolymer is comprised of at least one hydrophobic segment and at least one hydrophilic segment attached to said hydrophobic segment at its first end.

9. A dialyzer as in claim 8, wherein said hydrophobic membrane is a semipermeable polysulfone membrane or semipermeable polyethersulfone membrane.

10. A dialyzer as in claim 8, wherein said hydrophobic segment is PPO and said hydrophilic segment is PEO.

11. A dialyzer as in claim 8, wherein said copolymer has the chemical structure formula:

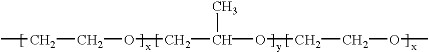

12. A dialyzer as in claim 11, wherein the x=129 and the y=56 in said formula.

13. A dialyzer as in claim 8, wherein at least one of said hydrophilic segments has a modified active group attached to it.

14. A dialyzer as in claim 13, wherein said modified active group is comprised of at least one of a metal-chelating end group, primary amine group or pyridyl disulfide group.

15. A method for coating a hydrophobic membrane for use in dialysis, hemodialysis, hemofiltration, hemodiafiltration or other end stage renal disease treatments comprising:
(1) dissolving at least one copolymer in water to form a solution, wherein said copolymer is comprised of at least one hydrophobic segment and at least one hydrophilic segment; and
(2) exposing said hydrophobic membrane to said solution so that said copolymer adheres to the surface of said hydrophobic membrane.

16. A method according to claim 15, wherein said hydrophobic membrane is a semipermeable polysulfone membrane or a semipermeable polyethersulfone membrane.

17. A method according to claim 15, wherein said hydrophobic segment is PPO and said hydrophilic segment is PEO.

18. A method according to claim 15, wherein said copolymer has the chemical structure formula:

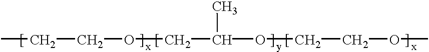

19. A method according to claim 18, wherein the x=129 and the y=56 in said formula.

20. A method according to claim 15, wherein at least one of said hydrophilic segments has a modified active group attached to it.

21. A method according to claim 20, wherein said modified active group is comprised of at least one of a metal-chelating end group, primary amine group or pyridyl disulfide group.

22. A method according to claim 15, wherein said water is reverse osmosis, deionized water.

23. A method according to claim 15, wherein said solution has a weight/volume ratio that is less than the critical gel point for said solution.

24. A method according to claim 15, wherein said hydrophobic membrane is exposed to said solution for more than 2 minutes.

25. A method according to claim 15, wherein said solution is brought to a temperature of greater than 20 degrees Celsius before exposing said hydrophobic membrane to said solution.

26. A method according to claim 15, wherein said hydrophobic membrane is inside a dialyzer and said hydrophobic membrane is exposed to said solution by pumping said solution through at least one compartment of said dialyzer.

27. A dialysis set for use in dialysis, hemodialysis, hemofiltration, hemodiafiltration or other end stage renal disease treatments comprising a set of tubing, dialysate solution container, at least one pump, and a dialyzer, wherein said dialyzer has a hydrophobic membrane, at least one copolymer is attached to said hydrophobic membrane, and each copolymer is comprised of at least one hydrophobic segment and at least one hydrophilic segment.

28. A dialysis set as in claim 27, wherein said hydrophobic membrane is a semipermeable polysulfone membrane or semipermeable polyethersulfone membrane.

29. A dialysis set as in claim 27, wherein said hydrophobic segment is PPO and said hydrophilic segment is PEO.

30. A dialysis set as in claim 27, wherein said copolymer has the chemical structure formula:

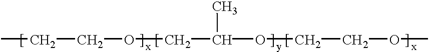

31. A dialysis set as in claim 30, wherein the x=129 and the y=56 in said formula.

32. A dialysis set as in claim 27, wherein at least one of said hydrophilic segments has a modified active group attached to it.

33. A dialysis set as in claim 32, wherein said modified active group is comprised of at least one of a metal-chelating end group, primary amine group or pyridyl disulfide group.

34. A method for reducing the adsorption and deposition of blood constituents on a hydrophobic membrane surface for use in dialysis, hemodialysis, hemofiltration, hemodiafiltration or other end stage renal disease treatments comprising:
(1) dissolving at least one copolymer in water to form a solution, wherein said copolymer is comprised of at least one hydrophobic segment and at least one hydrophilic segment attached to said hydrophobic segment; and
(2) exposing said hydrophobic membrane to said solution so that said copolymer adheres to the surface of said membrane.

35. A method according to claim 34, wherein said hydrophobic membrane is a semipermeable polysulfone membrane or a semipermeable polyethersulfone membrane.

36. A method according to claim 34, wherein said hydrophobic segment is PPO and said hydrophilic segment is PEO.

37. A method according to claim 34, wherein said copolymer has the chemical structure formula:

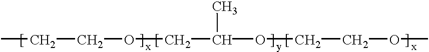

38. A method according to claim 37, wherein the x129 and the y=56 in said formula.

39. A method according to claim 34, wherein at least one of said hydrophilic segments has a modified active group attached to it.

40. A method according to claim 34, wherein said modified active group is comprised of at least one of a metal-chelating end group, primary amine group or pyridyl disulfide group.

41. A method according to claim 34, wherein said water is reverse osmosis, deionized water.

42. A method according to claim 34, wherein said solution has a weight/volume ratio that is less than the critical gel point for said solution.

43. A method according to claim 34, wherein said hydrophobic membrane is exposed to said solution for more than 2 minutes.

44. A method according to claim 34, wherein said solution is brought to a temperature of greater than 20 degrees Celsius before exposing said hydrophobic membrane to said solution.

45. A method according to claim 34, wherein said hydrophobic membrane is inside a dialyzer and said hydrophobic membrane is exposed to said solution by pumping said solution through at least one compartment of said dialyzer.

46. A method for performing dialysis, hemodialysis, hemoperfusion, hemodiafiltration or other end stage renal disease treatments using a dialyzer that contains a hydrophobic membrane, wherein at least one copolymer is attached to said hydrophobic membrane and each copolymer is comprised of at least one hydrophobic segment and at least one hydrophilic segment.

47. A coated membrane as in claim 46, wherein said hydrophobic membrane is a semipermeable polysulfone membrane or semipermeable polyethersulfone membrane.

48. A coated membrane as in claim 46, wherein said hydrophobic segment is PPO and said hydrophilic segment is PEO.

49. A coated membrane as in claim 46, wherein said copolymer has the chemical structure formula:

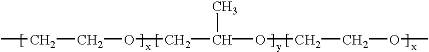

50. A coated membrane as in claim 49, wherein the x=129 and the y=56 in said formula.

51. A coated membrane as in claim 46, wherein at least one of said hydrophilic segments has a modified active group attached to it.

52. A coated membrane as in claim 51, wherein a said modified active group is comprised of at least one of a metal-chelating end group, primary amine group or pyridyl disulfide group.

53. A method for performing dialysis, hemodialysis, hemoperfusion, hemodiafiltration or other end stage renal disease treatments comprising:
(1) taking fluid from a patient;
(2) pumping said fluid through a dialysis set, wherein said dialysis set includes a dialyzer with a hydrophobic membrane, wherein at least one copolymer is attached to said hydrophobic membrane and each copolymer is comprised of at least one hydrophobic segment and at least one hydrophilic segment; and
(3) returning said fluid to said patient.

54. A method according to claim 53, wherein said hydrophobic membrane is a semipermeable polysulfone membrane or semipermeable polyethersulfone membrane.

55. A method according to claim 53, wherein said hydrophobic segment is PPO and said hydrophilic segment is PEO.

56. A method according to claim 53, wherein said copolymer has the chemical structure formula:

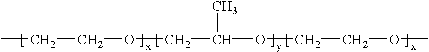

57. A method according to claim 56, wherein the x129 and the y=56 in said formula.

58. A method according to claim 53, wherein at least one of said hydrophilic segments has a modified active group attached to it.

59. A method according to claim 58, wherein said modified active group is comprised of at least one of a metal-chelating end group, primary amine group or pyridyl disulfide group.
